(19) United States
(12) Patent Application Publication
(10) Pub. No.: US 2008/0051768 A1
(43) Pub. Date: Feb. 28, 2008

(54) TRACKABLE MEDICAL INSTRUMENT COMPRISING AN EXCHANGEABLE TIP

(76) Inventor: Tanja Stumpf, Munchen (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/842,969

(22) Filed: Aug. 22, 2007

Related U.S. Application Data
(60) Provisional application No. 60/824,365, filed on Sep. 1, 2006.

(30) Foreign Application Priority Data
Aug. 22, 2006 (EP) 06017395

(51) Int. Cl. A61B 17/00

(52) U.S. Cl. 606/1

(57) ABSTRACT

A medical instrument having a handle and a functional portion includes a first part, a second part selectively couplable to the first part and a plurality of trackable markers arranged on the instrument. At least one marker is arranged on the first part and at least one marker is arranged on the second part.

### RELATED APPLICATION DATA

This application claims priority of U.S. Provisional Application No. 60/824,365 filed on Sep. 1, 2006, which is incorporated herein by reference in its entirety.

### FIELD OF THE INVENTION

The invention relates to medical instruments and, more particularly, to a trackable medical instrument that includes an exchangeable tip.

### BACKGROUND OF THE INVENTION

Medical instruments can be used in image-assisted surgery, wherein tracking markers attached to the instrument in conjunction with a medical tracking system enable the instrument to be tracked. Further, with the aid of a medical navigation system, the particular instrument (including its functional portion) may be identified, and the instrument may be localized and tracked in three-dimensional space. The medical instrument then can be displayed on an image output device relative to previously ascertained patient data (e.g., from imaging methods; CT, MR, etc.) in order to provide the surgeon with visual assistance in his work.

Medical instruments comprising tracking markers or tracking marker attachments are known, for example, from DE 196 39 615 A1.

In order to make such instruments, e.g., instruments comprising reflector marker arrays, unambiguously identifiable by a navigation system, a group of reference markers typically are assigned to each of the instruments, wherein the group of markers exhibits an arrangement that is characteristic for the particular instrument. This can make producing such instruments, including fitting them with groups of reference markers, elaborate and expensive, in particular when they are provided as a set of instruments.

### SUMMARY OF THE INVENTION

A medical instrument can be divided into at least two selectively couplable parts, wherein at least one tracking marker is arranged on each part. This divisibility means that an instrument part can be exchanged, which provides a way of influencing the arrangement of the tracking markers of the instrument. One or more tracking markers on one part of the instrument can then remain as they are, and when a substitute instrument part is combined with the remaining instrument part, a new marker array is created that is again characteristic for the new instrument. The medical instrument described herein thus dispenses with the need to provide complete new marker arrays for different instruments.

A first part (e.g., a distal part) of the instrument can include a functional portion, e.g., the instrument tip, which, depending on the instrument, can be configured differently from other distal parts, and may be suitable for different operations (e.g., a pointer tip, forceps, scalpel blade, etc.). A second part (e.g., a proximal part) of the instrument can include at least a part of a handle (e.g., the handle of the instrument can be divided into two or more parts).

At least one tracking marker or group of tracking markers can be arranged on the first part of the instrument, and the configuration or arrangement of the tracking marker or group of tracking markers is unambiguously assigned to the first part (e.g., to the functional portion of the instrument). A separate and distinguishable overall tracking marker array on the instrument thus results for each first part of the instrument (including the functional portion).

Further, at least one tracking marker or group of tracking markers can be arranged on the second part of the instrument. When the first and second instrument parts are combined, the overall geometry of the tracking marker or group of tracking markers of the first part together with the tracking marker or group of tracking markers of the second part is characteristic for one instrument composition and serves to individually identify such combination. Individual identification is ensured if, for identical geometries of the second part, the position of the tracking marker or the positions of the group of tracking markers differ at least in their axial position. Generally, a change in the axial and radial position can be used to create variable the geometries.

In the case of medical instruments comprising first and second parts as described herein, it is possible to arrange one tracking marker on the first instrument part and another tracking marker on the second instrument part. Alternatively, it is also possible to arrange one tracking marker on the first instrument part and a group of tracking markers, comprising two or more tracking markers, on the second instrument part. It is of course also possible for the first instrument part to comprise a number of tracking markers. In each instrument, the tracking marker or tracking markers for the first instrument part have a unique arrangement that can be assigned to the instrument part (e.g., the functional part).

The two instrument parts can be connected by a non-rotational connection. The connection can comprise one or more of the following types of connection means: a screw thread connection comprising positive-lock or friction-lock engaging means between the first and second instrument part; a latch connection comprising positive-lock or friction-lock engaging means between the first and second instrument part; a plug connection comprising positive-lock or friction-lock engaging means between the first and second instrument part.

As already briefly noted, the tracking markers of a medical instrument can serve for tracking the instrument by means of a medical tracking system and for identifying the instrument by a medical navigation system.

A set of medical instruments comprising at least two instruments such as have been described herein is also provided. “Two instruments” does not necessarily mean that two first instrument parts and two second instrument parts have to be provided; rather, the instruments can be constructed from various first instrument parts, each of which comprise the functional portion, and from the same second instrument part or identical second instrument parts that comprise at least a part of the handle. A single second instrument part, to which a number of first instrument parts can be exchangeably attached, is in principle sufficient to provide the set of instruments.

The instruments described herein enable a user, for example a surgeon, to adapt his instrument to the situational requirements. It is only necessary to exchange the one part of an instrument (e.g., the first part) in order to produce a completely different instrument, wherein the new first part (e.g., the distal part) including its reference marker or reference markers is identified, together with the second part (e.g., the proximal part) by the navigation system based on the configuration or arrangement of all the markers situated on the instrument. To this end, the various combinations can be stored in the navigation system. When a combination is identified, the newly combined instrument then can be unambiguously identified and assigned, and the navigation system no longer has to be separately informed as to which instrument is currently being used.

The instrument described herein can be easily adapted to the needs at hand. It is no longer necessary to provide a plurality of different complete instruments; one proximal part (second instrument part) and a collection of a number of distal parts is in principle sufficient. The geometry of the trackable passive marker arrays can be automatically identified by the navigation system.

Moreover, a medical instrument is provided in which the front part or tip is exchangeable. If, for example, two optical tracking markers are arranged on a standard second part (e.g., rear or proximal part), a third tracking marker can be arranged on the exchangeable first part (e.g., front or distal part). The distance from the third marker to a point on the standard second part (e.g., a marker) or also to a part of the instrument itself (facing side) and/or its height above the axis of its standard second part, form a characteristic and unique geometry of the tracking markers. One of the two measures or both measures in combination (distance and height) can be different for each exchangeable first part and can therefore be unambiguously assigned.

### BRIEF DESCRIPTION OF THE DRAWINGS

The forgoing and other features of the invention are hereinafter discussed with reference to the drawing.

FIGS. 1 and 2 illustrate exemplary instruments in accordance with the invention, comprising a straight instrument tip and two or three tracking markers.

FIGS. 3 and 4 illustrate exemplary instruments in accordance with the invention, comprising a curved tip and two or three tracking markers.

FIGS. 5 and 6 illustrate exemplary instruments in accordance with the invention, comprising two or three tracking markers.

FIG. 7 illustrates an exemplary connector for the two instrument parts in accordance with the invention

FIG. 8 illustrate another exemplary connector for the two instrument parts in accordance with the invention.

### DETAILED DESCRIPTION

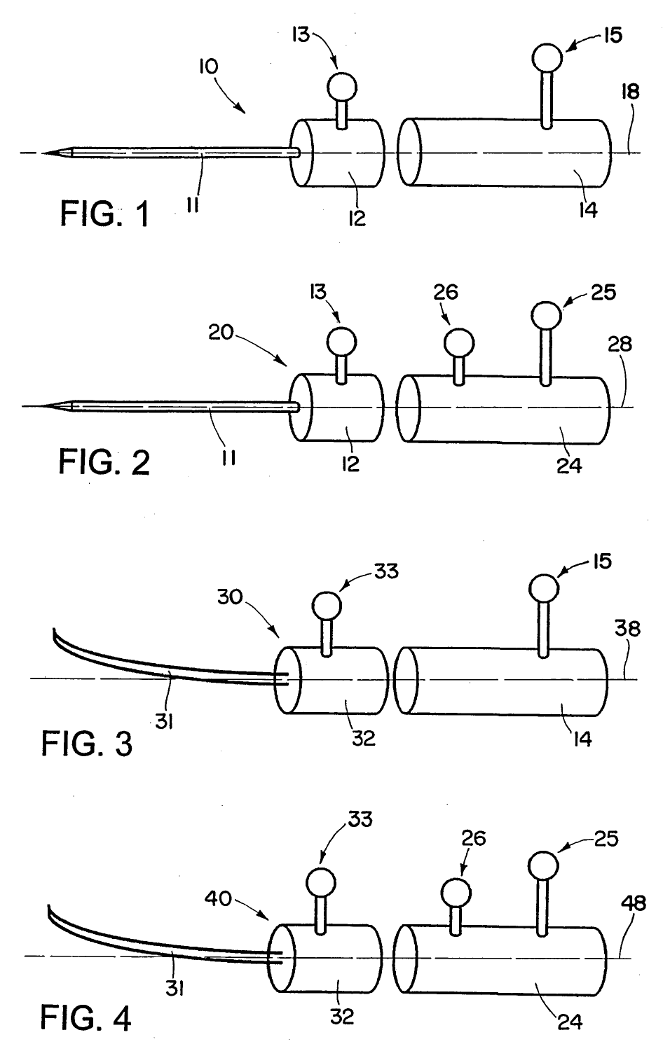

FIGS. 1 and 2 show exemplary instruments 10 and 20 that include the same first part 11, but different second parts 14 and 24, respectively. The instruments 10 and 20 are shown divided in the figures so as to illustrate the first and second parts. In use, the instrument parts are of course connected to each other.

The instruments 10 and 20 comprise straight instrument tips 11 which form the front portion of the distal part 12. Passive reflection markers 13, for example, are attached to the distal parts 12 at a mid point from a longitudinal axes 18 and 28 of the respective instruments 10 and 20. The distal parts 12 can be connected to proximal parts 14 (FIG. 1) and 24 (FIG. 2), wherein the proximal part 14 comprises a single tracking marker 15, and the proximal part 24 comprises two tracking markers 25 and 26.

In the case of the instruments according to FIG. 1, the characteristic configuration of the marker array can be determined by the distance between the markers 13 and 15. In the case of the instrument 20 according to FIG. 2, the characteristic arrangement can be determined by the relative distances between the three markers 13, 25 and 26.

FIGS. 3 and 4 show exemplary instruments 30 and 40 that include bent tips 31. The instruments 30 and 40 can comprise proximal parts 14 and 24, which are identical to the proximal parts 14 and 24 of FIGS. 1 and 2. In order to be unambiguously identified, however, the instruments 30 and 40 shown in FIGS. 3 and 4 differ from the instruments 10 and 20 with regard to the arrangement of the tracking marker 33 on the distal parts 32. A distance from the markers 33 to the longitudinal centerline 38 and 48 of the respective instruments 30 and 40 is greater than a distance from the markers 13 to the longitudinal centerline 18 and 28 of the respective instruments 10 and 20. Moreover, the marker configurations as a whole (marker on the proximal part plus markers on the distal part) can be distinguished from other marker configurations (e.g., those for the instruments 10 and 20) by this different measure. On the basis of these different marker configurations, a navigation system can identify that the instruments are currently the instruments 30 and 40, respectively, even though the proximal parts 14 and 24 of the instruments 30 and 40 are the same as the proximal parts 14 and 24 of the instruments 10 and 20.

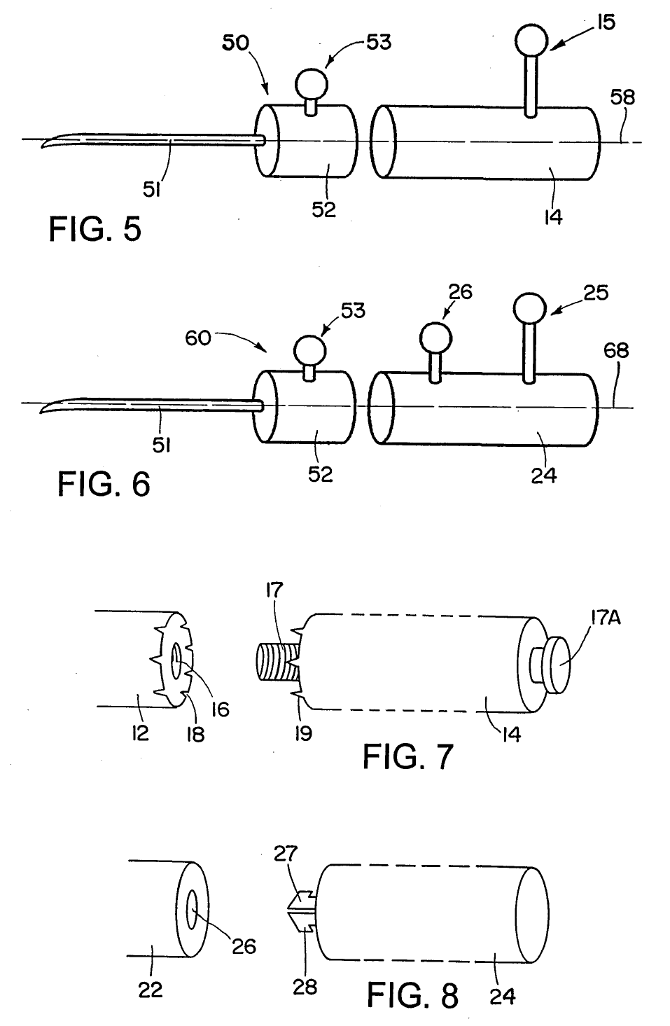

Similarly, FIGS. 5 and 6 illustrate exemplary instruments 50 and 60 having identical distal parts 52 with a short, curved tip 51, wherein markers 53 exhibit only a very short distance from the longitudinal axis 58 and 68 of the respective instruments 50 and 60. On the basis of this short distance (and the altered overall marker configuration for the instrument), each instrument 50 and 60 can be unambiguously identified.

Accordingly, it is possible to combine the proximal part 14 with one of the distal parts 12, 32 and 52 so as to obtain three different, unambiguously identifiable instruments 10, 30 and 50. Similarly, it is possible to combine the proximal part 24 with one of the distal parts 12, 32 and 52 so as to obtain another three different, unambiguously identifiable instruments 20, 40 and 60.

FIG. 7 shows an exemplary connector means for the instrument 10 of FIG. 1, wherein the connector means couples the distal part 12 to the proximal part 14. The exemplary distal part 12 comprises a centric threaded bore 16 and notch-like cavities 18 on the circumference of its facing side. Matching the bore 16 and cavities 18, the proximal part 14 comprises a threaded rod 17 which can rotate within the proximal part 14 and is connected to the actuator 17A by which the threaded rod 17 can be rotated. Projecting links 19 are provided on the circumference of the proximal part 14 at its end where the threaded rod exits the proximal part 14. When the two parts 12 and 14 are assembled, the threaded rod 17 is screwed into the inner thread 16, and the links 19 come to rest in the notches 18. Coupling the parts by means of the actuator 17A ultimately creates a non-rotational connection that ensures that the markers remain in the correct arrangement, i.e., in the arrangement stored in the navigation system.

FIG. 8 shows another exemplary connector means with respect to the instrument 20 of FIG. 2, wherein the distal part 22 comprises a centric bore 26 into which a locking protrusion 27 of the proximal part 24 can lock. The outwardly pointing ribs 28 prevent the parts 22 and 24 from rotating relative to each other and also prevent said two parts from detaching from each other. A device could also be provided, for example, that causes the ribs 28 to collapse or fold so as to enable the two parts to be selectively separated from each other. It is also advantageous if an aligning aid, for example a mechanical engagement or an indicator, is provided on the first and/or second part that ensures the connection be made in the correct rotational position of the two parts.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A medical instrument including a handle and a functional portion, comprising:
a first part;
a second part selectively couplable to the first part; and
a plurality of trackable markers arranged on the instrument, wherein at least one marker of the plurality of markers is arranged on the first part and at least one other marker of the plurality of markers is arranged on the second part.

2. The medical instrument according to claim 1, wherein the first part includes the functional portion, and the second part includes at least part of the handle.

3. The medical instrument according to claim 2, wherein a configuration or arrangement of the at least one marker on the first part is uniquely assigned to the functional portion of the instrument.

4. The medical instrument according to claim 3, wherein the at least one marker arranged on the first part is a group of markers.

5. The medical instrument according to claim 3, wherein the assignment of the at least one marker arranged on the first part is determined by a characteristic arrangement assigned to the functional part relative to a marker geometry of the second part.

6. The medical instrument according to claim 1, wherein the at least one other marker arranged on the second part is a group of markers.

7. The medical instrument according to claim 1, further comprising a coupling, wherein at least one part of the coupling is arranged on the first part, and at least one other part of the coupling is arranged on the second part, said coupling operable to maintain an orientation of the first part relative to the second part.

8. The medical instrument according to claim 7, wherein the coupling comprises at least one of:
a screw thread connection including a positive-lock or friction-lock engaging device arranged between the first and second part;
a latch connection including a positive-lock or friction-lock engaging device arranged between the first and second part; or
a plug connection including a positive-lock or friction-lock engaging device arranged between the first and second part.

9. The medical instrument according to claim 1, wherein the plurality of markers are operative to provide data indicative of a location of the markers in three-dimensional space to a medical tracking system so as to enable the instrument to be identified by a medical tracking system from a group of different medical instruments.

10. A set of medical instruments comprising at least two instruments according to claim 1.

11. The set of medical instruments according to claim 10, wherein the set of instruments is constructed from various first parts each of which comprise a functional portion, and from the same second part that comprises at least a part of the handle.
